(19) United States
(12) Patent Application Publication
(10) Pub. No.: US 2006/0068399 A1
(43) Pub. Date: Mar. 30, 2006

(54) Multiple bead reagent system for protein based assays with optimized matrices

(75) Inventors: William A. McMillan, Cupertino, CA (US); Byung Sook Moon, Palo Alto, CA (US); Martin Jones, Walnut Creek, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cepheid, Sunnyvale, CA (US)

(21) Appl. No.: 10/949,266

(22) Filed: Sep. 24, 2004

(51) Int. Cl. C12Q 1/68; G01N 33/53

(52) U.S. Cl. 435/6; 435/7.1

(57) ABSTRACT

The invention provides a multi-bead assay system for a protein based assay comprising at least two different beads. The first bead comprises protein and a protein stabilization matrix. The first bead forms a first solution when dissolved in liquid, and the first solution permits a first activity level for the assay. The second bead comprises a potentiation bead matrix that when dissolved in the first solution forms a second solution that potentiates the protein based assay to achieve a second activity level that is higher than the first activity level.

### CROSS-REFERENCES TO RELATED APPLICATIONS

Not applicable

### STATEMENT AS TO RIGHTS TO INVENTIONS MADE UNDER FEDERALLY SPONSORED RESEARCH OR DEVELOPMENT

Not applicable

### REFERENCE TO A “SEQUENCE LISTING,” A TABLE, OR A COMPUTER PROGRAM LISTING APPENDIX SUBMITTED ON A COMPACT DISK

Not applicable

### FIELD OF THE INVENTION

The invention provides a multi-bead assay system for a protein based assay comprising at least two different beads.

### BACKGROUND OF THE INVENTION

Diagnostic assays for environmental quality, forensics and the diagnosis of disease frequently employ enzymes, antibodies, and other water-soluble proteins. To safeguard the shelf life and accuracy of these diagnostic tests, the proteins must be kept stable and viable. Unfortunately, protein reagents for protein based assays may be subject to significant losses of activity, physicochemical changes, or degradation both during storage and in solution prior to the actual start of an assay. Since degradation and loss of activity can affect the outcome of experimental results, it is essential to both monitor and control the stability of proteins used in protein-based high throughput diagnostic assays.

Naturally, enzymes, antibodies, and the like would be more economical if they were stable for long periods of storage since reagents could more confidently be purchased in bulk. Unfortunately, conditions that may be optimal for storage of protein reagents may not be optimal for the biological reaction. Indeed, compounds and excipients added to facilitate optimal storage may even inhibit the intended biological reaction. Thus, there is a need in the art for stabilization reagents that permit increased shelf life without negatively interfering with the biological activity of the protein.

Surprisingly, it has been found that by combining the protein reagents with specific additives in accordance with the invention, it is possible to formulate compositions that the increase the stability of proteins under conditions of storage, but which do not inhibit biological activity. Furthermore the invention provides means for potentiating the biological activity of the stored reagent beads once they are in solution. Thus, the present invention is uniquely designed so that the labile protein reagents in are effectively “stabilized” under conditions of storage so that their activity may be potentiated in solution.

### SUMMARY

The invention provides a multi-bead assay system for a protein based assay. The assay system comprises: a first bead that comprises a protein and a protein stabilization matrix and which forms a first solution when dissolved in liquid. The first solution permits a first activity level for the assay. The assay system also includes a second bead comprising a potentiation bead matrix that when dissolved in the first solution forms a second solution that potentiates the protein based assay to achieve a second activity level that is higher than the first activity level. The activity level, when it is above zero means that the reaction has all the active ingredients needed for the reaction to proceed. The active ingredients may be supplied entirely by the bead or by a combination of the first bead and the liquid.

In one embodiment, the second solution potentiates the protein based assay at least 2-fold over the first activity level of the first solution, and more preferably five fold. In some embodiments, the protein based assay is selected from the group consisting of an enzymatic assay, an antibody based assay, and a receptor based assay. In some embodiments, the protein based assay comprises nucleic acid amplification, the first bead comprises a lyophilized reagent bead containing at least one enzyme for the nucleic acid amplification, and the second bead comprises a lyophilized reagent bead containing primers for amplification of at least one, sometimes two, or sometimes three or more analyte nucleic acid sequences. In some embodiments, the second bead further comprises probes for detection of the analyte nucleic acid sequences.

According to another aspect, the invention provides a multi-bead assay system for a protein based assay, the assay system comprising: a first bead and a second bead, wherein the first bead comprises a bead that yields a first solution of a first pH when the bead is dissolved in liquid, and the second bead comprises a bead that yields a second solution of a second pH when the bead is dissolved in liquid. The difference in pH between the first solution and the second solution is at least 0.4 pH units. In one embodiment, combining the first solution and the second solution results in a third solution of a third pH that permits a protein based assay to take place at an activity level that is greater than an activity level of the protein based assay at the first pH. In some embodiments, the protein based assay comprises nucleic acid amplification, the first bead comprises a lyophilized reagent bead containing at least one enzyme for the nucleic acid amplification, and the second bead comprises a lyophilized reagent bead containing primers for amplification of at least one, sometimes two, or sometimes three or more analyte nucleic acid sequences. In some embodiments, the second bead further comrpises probes for detection of the analyte nucleic acid sequences.

The invention also provides a multi-bead reaction system for nucleic acid amplification comprising a first lyophilized reagent bead comprising at least one enzyme for nucleic acid amplification in a protein stabilization matrix, and a second lyophilized reagent bead comprising oligonucleotides for nucleic acid amplification in a potentiation bead matrix, wherein combining and dissolving the reagent beads in water potentiates the nucleic acid amplification reaction. In one embodiment, the multi-bead reaction system further comprises a means for detecting amplification products, such as an intercalating agent in the second bead or one or more hybridization probes in the second bead. In some embodiments, the second lyophilized reagent bead comprises primer oligonucleotides and probe oligonucleotides for amplification and detection of one or more analyte nucleic acid sequences. In another embodiment the multi-bead reaction system further comprises a third bead that comprises an oligonucleotide probe for detection of nucleic acid amplification product. In another embodiment, the third bead further comprises an intercalation agent, such as SYBR-green®.

In some embodiments, the nucleic acid amplification reaction is an isothermic amplification reaction selected from the group consisting of strand displacement amplification, transcription mediated amplification, rolling circle amplification and nucleic acid sequence based amplification. In other embodiments, the nucleic acid amplification reaction is a thermocyclic amplification reaction selected from the group consisting of polymerase chain reaction (PCR), reverse transcription polymerase chain reaction (RT-PCR), and ligase chain reaction (LCR).

### BRIEF DESCRIPTION OF THE DRAWINGS

FIG. 1. 4-Plex Reagent Stability using GeneXpert End Point Fluorescence (pX01) at 45° C. Positive Control

FIG. 2. 4-Plex Reagent Stability on GeneXpert using End Point Fluorescence (pX02) at 45° C. Positive Control

FIG. 3. Plex Reagent Stability using GeneXpert End Point Fluorescence (internal control) at 45° C. Positive Control

FIG. 4. Ba 4-Plex Reagent Stability using GeneXpert End Point Fluorescence (sample preparation control) at 45° C. Positive Control

FIG. 5. Ba Simplex assay—Ba DNA concentration vs. Cycle Threshold.

FIG. 6. FIG. 6; Ba Simplex assay—Ba DNA concentration vs. End Point Flouresence (pX01) Real Time PCR.

FIG. 7. Ba Duplex assay Ba DNA concentration vs. Cycle Threshold.

FIG. 8. Ba Duplex assay—Ba DNA concentration vs. End Point Flouresence (pX01) Real Time PCR.

FIG. 9. Ba 4-Plex assay—Ba lysed spore concentration vs. Cycle Threshold

FIG. 10. Ba 4-Plex assay—Ba lysed spore concentration vs. End Point Flouresence (pX01) Real Time PCR.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, analytical chemistry, and nucleic acid chemistry and hybridization described below are those well known and commonly employed in the art. The techniques and procedures are generally performed according to conventional methods in the art and various general references (see generally, Kochanowski, et. al., eds. Quantitative PCR Protocols (Methods in Molecular Medicine, Vol 26), Humana Press: Totowa, N.J., (1999), which is incorporated herein by reference), which are provided throughout this document. Standard techniques, or modifications thereof, are used for chemical syntheses and chemical analyses.

The phrase “multi-bead assay system” refers to an assay system for detecting the presence or absence of a particular target molecule or reagent, wherein the components of the assay system are contained within more than one matrix, and each matrix has the form of a lyophilized bead.

A “bead”, as used herein, refers to a small, compact form that often, but not always, has a spherical or nearly spherical, e.g., elliptical, shape. In an exemplary embodiment, the beads have cross-sections which are between one millimeter and twenty-five millimeters. In another exemplary embodiment, the beads have cross-sections which are between five millimeters and fifteen millimeters. In yet another exemplary embodiment, the beads have cross-sections which are between one millimeter and six millimeters. In still another exemplary embodiment, the beads have cross-sections which are between one millimeter and four and a half millimeters.

The expression “a protein based assay” refers to any method of analyzing, quantitating or otherwise reacting substances that employs proteins as active agents. Thus, the term “enzymatic assay” refers to a protein based assay wherein the active protein functions as a catalyst. Examples of enzymatic assays include restriction digests, and nucleic acid amplification reactions. Similarly, the term “antibody based assay” refers to an assay where the active protein is an antibody. For example, an ELISA assay would be an “antibody based assay”. The term “receptor based assay” refers to an assay that employs a receptor protein such as the acetylcholine receptor, the insulin receptor or a glucocorticoid receptor in a binding assay where the binding of a ligand to the receptor is measured in the course of the assay.

The term “protein stabilization matrix” refers to compounds and chemicals of a lyophilized reagent bead comprising reagents that stabilize the protein components of the reagent bead. The components of the protein stabilization matrix may include, but are not limited to: buffering agents such as HEPES, sugars such dextrose and trehalose; polyols such as glycerol, mannitol, sorbitol, xylitol; salts especially salts comprising ammonium (NH₄⁺), and sulphate (SO₄²⁺) ions, citrates, acetates; quaternary ions generally such as sulphate and phosphate ions; amino acids especially glycine and alanine, at lower pH values also glutamate and aspartate, and lysine/EDTA; fatty acids, surfactants such as TWEEN 20; chelating agents; reducing agents such as DTT (dithiothreatol), and bulking agents. The protein stabilization matrix can be optimized to stabilize the active ingredients in their dry form so as to permit extended periods of dry storage. In addition the protein stabilization matrix can be optimized to achieve functionality of the active ingredients in solution.

The term “potentiation bead matrix” refers to a bead comprising ingredients which when combined with the ingredients of the protein stabilization matrix beads demonstrably potentiate the protein based reaction.

A protein reagent of the invention is “stable” or “stabilized” if it exhibits good stability as determined by a stability test as described herein.

The term “active ingredient” refers to substances that play a critical and direct role in the ability of a reaction to proceed from reactants to products. For example, active ingredients of a PCR reaction may include the polymerase enzyme, enzyme cofactors such as Mg²⁺, nucleic acid template molecule(s), primers, probes or labeled probes and the enzymes for detection of the labeled probes, NTPs, and vitamins. Active ingredients for other enzymatic protein based assays may also include the enzyme, and enzyme cofactors such as ATP or NAD. For receptor based assays active ingredients may further include a receptor protein, and receptor ligands or labeled ligands.

The term “passive ingredient” refers to substances that may facilitate or enhance the performance of a protein based reaction, but which are not essential for the reaction to take place. Unlike “inert” or “inactive” ingredients such as protein stabilization components, passive ingredients facilitate the reaction by indirectly participating in it. For example, KCl or NaCl may be added to a PCR reaction to facilitate primer annealing. The salts participate directly in the reaction in that they facilitate the hybridization step of the PCR. However, KCl and NaCl are not essential for the PCR reaction as the reaction would proceed, albeit less efficiently, in the absence of the added salts.

A “probe” refers to a molecule that allows for the detecting of the polynucleotide sequence of interest. In certain embodiments, a probe comprises a polynucleotide sequence capable of hybridization to a polynucleotide sequence of interest. In other embodiments, a probe comprises an agent capable of intercalating into a polynucleotide sequence of interest. Examples of intercalating agents include ethidium bromide or SYBR Green. In other embodiments, the probe comprises a label. The probes are typically labeled either directly, as with isotopes, chromophores, lumiphores, chromogens, or indirectly, such as with biotin, to which a streptavidin complex may later bind. Thus, the labels of the present invention can be primary labels (where the label comprises an element that is detected directly or that produces a directly detectable element) or secondary labels (where the detected label binds to a primary label, e.g., as is common in immunological labeling). In some embodiments, labeled nucleic acid probes are used to detect hybridization. Nucleic acid probes may be labeled by any one of several methods typically used to detect the presence of hybridized polynucleotides. In some embodiments, label detection occurs through the use of autoradiography with ³H, ¹²⁵I, ³⁵S, ¹⁴C, or ³²P-labeled probes or the like. Other labels include, e.g., ligands which bind to labeled antibodies, fluorophores, chemiluminescent agents, intercalating agents, enzymes, and antibodies which can serve as specific binding pair members for a labeled ligand. An introduction to labels, labeling procedures, and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

The term “inert ingredient” refers to substances that do not directly or indirectly participate in the reaction of the “active” ingredients. “Inert” ingredients may facilitate a reaction in that they may stabilize an “active” ingredient in the dry state, but they do not participate in the reaction itself. For example, amino acids, carbohydrates, or chelating agents may facilitate a protein based reaction because they stabilize the active protein reagent during storage, thereby increasing the time over which the active ingredient maintains its biological activity.

The term “internal control” as used herein, refers to a control reaction run in parallel, in the same container, and under the same conditions as a reaction of interest, that functions as a standard of comparison that is able to account for and sometimes adjust for extraneous influences on the reaction of interest.

The term “activity” refers to the actual or potential ability of a substance or set of substances to react, relative to some standard state. “Activity” may be a reaction rate, a concentration, a partial pressure, release of chemical potential, or any other unit of measurement appropriate to the reaction or substance in question.

The “activity level” refers to the ability of a substance or set of substances to react relative to the reactive potential under optimal, defined conditions. For example, if the maximum rate of a reaction is 10⁻⁵ mol sec⁻¹ and the present rate of that reaction is 10⁻⁹ mol sec⁻¹ the activity level of the reaction is “low” relative to the maximum possible reaction rate. Changing the reaction conditions so as to facilitate the reaction and increase the reaction rate from its present 10⁻⁹ mol sec⁻¹ to 10⁻⁸ mol sec⁻¹ would be said to have increased the activity level of the reaction 10-fold. If there is no detectable activity below or above the standard state, then the activity level can be said to be zero. The activity level when it is above zero means that the reaction has all the active ingredients needed for the reaction to proceed. The active ingredients may be supplied entirely by the bead or by a combination of the first bead and the liquid.

The term “potentiates” as used herein means to increase the actual or potential ability of a reaction to take place. For example, if a reaction of A+B leads to product C, and potentiator P, facilitates, but is not required for the reaction, then adding P to a solution containing A+B will “potentiate” the reaction. If the reaction of A+B requires another ingredient, I, to go to completion, the ingredient I does not “potentiate” the reaction because the reaction of A+B cannot take place without ingredient I. Thus, “potentiate” means that an ingredient has been added to the reaction mixture such that when all essential components of the reaction are present and appropriate conditions are applied to allow the reaction to take place, the reaction can proceed more efficiently, or more robustly, or to a greater extent than it would proceed in the absence of the potentiator, P. Under this definition, we exclude from a potentiator any active ingredient that is otherwise present in the reaction with the first bead in a rate limiting concentration where the potentiation bead provides additional amounts of the active ingredient. But the invention does include such beads with active reagents (ie. primers for PCR—active ingredient) where the potentiating bead also includes potentiating reagents such as buffers that optimize pH for the assay.

### DETAILED DESCRIPTION OF THE INVENTION

### I. Introduction

The invention provides a multi-bead assay system for a protein based assay that comprises a first bead, which comprises a protein and a protein stabilization matrix, wherein the protein stabilization matrix forms a first solution when dissolved in liquid and the first solution permits a first activity level for the assay where the first activity level is greater than zero. The invention further comprises a second bead that comprises a potentiation bead matrix, and when the potentiation bead matrix is dissolved in the first solution, it forms a second solution that potentiates the protein based assay to achieve a second activity level that is higher than the first activity level. In other words, the invention permits one to optimize protein stability in the first bead and optimize reaction conditions in the second bead. In one embodiment, the second solution potentiates the protein based assay at least 2-fold over the first activity level of the first solution. The protein based assay is selected from the group consisting of an enzymatic assay, an antibody based assay, and a receptor based assay.

The multi-bead assay system of the invention provides matrices that function to increase the stability of the protein reagents of the system under storage conditions. Improved stability increases the shelf life of the reagent bead and also increases the accuracy and quality of the protein based assay. Hence, the invention provides economic advantages over other currently available diagnostic systems employing protein based assays.

A major challenge to the formulation of reagent beads for protein based assays is to ensure the stability of the proteins as well as other biological reagents, over the expected shelf life of the reagent bead. Instability of proteins and peptides may be brought about through either physical or chemical instability. Physical instability may come about through a change in the secondary, tertiary or quaternary structure of the protein or peptide and may occur through processes such as denaturation, aggregation, precipitation and/or adsorbtion to surfaces. Chemical instability may come about through covalent modification of the protein via bond formation or cleavage occurring through reactions such as hydrolysis, deamination, oxidation, disulfide exchange, β-elimination, and racemation. Other biological reagents comprising reagent beads may also be subject to instability, but because the physical and chemical nature of these other reagents is different than that of the protein reagents, optimal conditions for storage of these reagents may differ.

It has now been discovered that reagent beads can be formulated to stabilize protein reagents in one bead, and other biological reagents in other beads. Upon mixing of the reagent beads in liquid, the stabilization matricies form a solution that provides optimal reaction conditions for the protein based assay.

Because all reaction components are stably formulated into reagent beads, the multi-bead assay system of the invention not only better stabilized biological reagents, it also increases the speed and accuracy of protein based diagnostic assays by minimizing the number of step-wise manipulations involved in setting up the reaction. Thus, the potential for error due to inaccuracies of individual measurement of each reagent and carry-over contamination is reduced or eliminated.

The types of protein based assays that will benefit from the formulations and methods of the invention include enzymatic assays such as PCR, antibody based assays such as ELISA, and receptor based assays such as fluorescence detection assays to determine ligand-receptor binding.

As the methods of the invention include the stabilization of assay reagents, methods and ingredients effective for the stabilization of biological reagents are set forth. The invention provides means for stabilizing proteins for increased shelf life. The invention also provides means for precisely controlling reaction conditions such that the potential biological activity of the proteins is controlled and brought forth only when desired. Thus, illustrative compounds and compositions which can be used in the reagent beads to achieve these effects are set forth, but these illustrations are not meant to be limiting. Since multiple lyophilized reagent beads comprise the assay system of the invention, routine procedures for the manufacture of lyophilized reagent beads are also set forth. Finally, exemplary protein based assays that could benefit from practice of the methods of the invention are disclosed. Thus, the specification provides means for making and using assay systems suitable for the practice of the invention.

### II. Formulation of the Matrices of the Invention

A. Introduction

Protein reagents for protein based assays may be subject to significant losses of activity, physicochemical changes, or degradation during storage. Since degradation and loss of activity can affect the outcome of experimental results, the rate of deterioration of the protein during storage is a useful parameter to measure. Lack of stability can mean rapid loss of biological activity or imprecise control over the reaction. Therefore protein stability is critical to obtaining reliable results.

According to the invention, reagent beads can be formulated so as to enhance the stability of proteins and other biological reagents during storage, and at the same time to ensure that upon dissolution of the reagent beads in liquid, optimal conditions are provided under which the protein based assay can proceed.

Stabilization of the protein reagents means that costs are reduced. Reagent beads comprising stabilized proteins provide products with increased shelf life, so bulk purchases of reagents may be obtained and stored without concern for loss of activity. Furthermore, stable preformulation of assay reagents ensures that all assay components are stabilized according to their individual requirements so that accurate and reproducible assay results can be achieved. Thus, in providing compositions comprising stabilized biological reagents, the invention provides economical protein based assay systems for diagnostic, forensic and environmental assays.

B. Matrix Formulation

The protein stabilization matrix comprises ingredients that function to stabilize the protein reagent in the dry state. Proteins comprising the reagent bead are contained within the structure of the protein stabilization matrix. The protein stabilization matrix may also contain within it preservatives and excipients as discussed in a later section of the specification.

1. Excipient

Excipients can be added to the reagent beads to facilitate bead formation, enhance buffering capacity, enhance protein solubility, or for any other reason for which they are appropriate. A large number of excipients are known to those of skill in the art and can comprise a number of different chemical structures. Examples of excipients, which may be used in the present invention, include carbohydrates, such as sucrose, glucose, trehalose, melezitose, dextran, and mannitol; proteins such as BSA, gelatin, and collagen; and polymers such as PEG and polyvinyl pyrrolidone (PVP). The total amount of excipient in the lyophilized bead may comprise either single or multiple compounds.

Excipients are added to reagent formulations for a variety of reasons, and each excipient has its own advantages and disadvantages. Thus, usually more than one excipient is required in the formulation to provide all the desirable attributes. For example, an excipient may be added to a formulation to be freeze dried so as to reduce the time for reconstitution. (see, e.g., Carpenter and Crowe, “The Mechanism of Cryoprotection of Proteins by Solutes,” Cryobiology, 25: 244-255 (1988)). Alternatively, excipients may be added to the formulation to facilitate attainment of the shape of the final lyophilized product. For example, excipient can be added to facilitate or prevent the product from attaining a bead like shape.

The type of excipient may also be a factor in controlling the amount of bead hygroscopy. Lowering bead hygroscopy can enhance the bead's integrity (accuracy of weighing beads) and cryoprotectant abilities. However, removing all water from the bead would have deleterious effects on those reaction components, proteins for example, that require certain amounts of bound water in order to maintain proper conformations. In general, the excipient level in the beads should be adjusted to allow moisture levels of less than 3%.

Naturally, there are limits to the amount of excipient which can be added to form a bead. If the amount of excipient is too low, the material does not coalesce to form a bead-like shape. At the high end, excipient amounts are limited by the solubility of the excipient in the bead buffer formulation. The amount is also dependent upon the properties of the excipient. In an exemplary embodiment, trehalose is present from between 5% to 20% (w/v). In another exemplary embodiment, mannitol is present from between 2% to 20% (w/v). In yet another exemplary embodiment, mannitol is present from between 2% to 20% (w/v) and dextran is present from between 0.5% to 5% (w/v). In still another exemplary embodiment, mannitol is present in the lyophilized bead in a weight percentage of between 40% to 75% (w/w).

Buffer

Exemplary buffers that may be employed, include, e.g., HEPES, borate, phosphate, carbonate, barbital, Tris, etc. -based buffers. See Rose et al., U.S. Pat. No. 5,508,178. The pH of the reaction should be maintained in the range of about 4.5 to about 9.5. See U.S. Pat. No. 5,508,178. The standard buffer used in amplification reactions is a Tris based buffer between 10 and 50 mM with a pH of around 8.3 to 8.8. See Innis et al., supra.

One of skill in the art will recognize that buffer conditions should be designed to allow for the function of all reactions of interest. Thus, buffer conditions can be designed to support the amplification reaction as well as any enzymatic reactions associated with producing signals from probes. A particular reaction buffer can be tested for its ability to support various reactions by testing the reactions both individually and in combination.

Salt Concentration

The concentration of salt present in the lyophilization mixture can be added to affect the ability of primers to anneal to the target nucleic acid in a nucleic acid amplification reaction. See Innis et al. Potassium chloride is typically added to so as to achieve up to a concentration of about 50 mM or more in the final solution upon reconstitution. Sodium chloride can also be added to promote primer annealing. See Innis et al. supra.

Carrier Proteins

Carrier proteins useful in the present invention include but are not limited to albumin (e.g., bovine serum albumin) and gelatin.

2. Biological Reagents

The present invention provides active ingredients comprising biological reagents that are required for protein based assays. In certain embodiments, the present invention can be used in nucleic acid amplification reactions. In other embodiments the invention can be useful for the practice of enzyme kinetic assays, and antibody or receptor-ligand binding assays. The active ingredients comprising the reagents beads include, but are not limited to proteins, nucleic acids, nucleotides, some minerals, and vitamins.

Proteins

In one aspect, the lyophilized bead may comprise an enzyme such as a DNA polymerase (e.g. Taq polymerase). For example, Taq DNA Polymerase may be used to amplify target DNA sequences. The amplification assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. In some embodiments, concentration ranges of the polymerase typically range from 1-12 units per reaction mixture. The reaction mixture is typically between 20 and 100 μL.

In some embodiments, a “hot start” methodology can be used in an amplification reaction to prevent extension of mispriming events as the temperature of a reaction initially increases. Hot starts are particularly useful in the context of multiplex PCR. Examples of hot start methodologies include heat labile adducts attached to a polymerase or ligase requiring a heat activation step (typically 95° C. for approximately 10-15 minutes) or an antibody associated with the polymerase or ligase to prevent activation.

In other aspects an RNA polymerase, or reverse transcriptase, or an enzyme such as tyrosine kinase may be used in the protein based assay. In still other aspects, the lyophilized reagent bead may contain an antibody, and in other embodiments the lyophilized reagent bead may contain a receptor such as Interleukin I, or Angiotensin II.

In addition, proteins such as those that facilitate detection of labeled probes, may be included as active ingredients in the reagent bead.

Nucleic Acids

The lyophilized reagent beads may also comprise active ingredients such as nucleic acids or nucleic acid precursors. For example, the lyophilized reagent beads may contain DNA templates that serve as controls for a nucleic acid amplification reaction. The lyophilized beads may also contain deoxynucleotide triphosphates (e.g., dATP, dCTP, dTTP, dGTP). When required, deoxynucleoside triphosphates (dNTPs) are added to the reaction to a final concentration of about 20 μM to about 300 μM. Each of the four dNTPs (G, A, C, T) are generally present at equivalent concentrations (See Innis et al supra). In some embodiments, the reaction mixtures of the invention will comprise oligonucleotide primers which hybridize to a particular DNA sequence of interest, or probes which can detect the presence of primer hybridization with the DNA sequence of interest.

Cofactors

The lyophilized beads may also comprise any number of cofactors that are essential active ingredients of the protein based assay. For example, nucleotides such as ATP or NAD, vitamins, or certain minerals may serve a cofactors in protein based assays. In particular, magnesium may be an important cofactor, whose concentration must be carefully balanced when used in thermocyclic amplification reactions that utilize Taq polymerase.

Magnesium Ion Concentration

As noted above, the concentration of magnesium ion can be critical in amplification reactions. Primer annealing, strand denaturation, amplification specificity, primer-dimer formation, and enzyme activity are all examples of amplification reaction parameters that are affected by magnesium concentration (see Innis et al.). The optimal magnesium concentration for a given amplification reaction can vary depending on the nature of the target nucleic acid(s) and the primers being used, among other parameters, and can be determined for a particulaer target nucleic acid primer combination by carrying out a series of amplification reactions over a range of magnesium concentrations to determine the optimal magnesium concentration. Typically the final concentration of magnesium in amplification reactions can be e.g., about a 0.5 to 2.5 mM magnesium concentration excess over the concentration of dNTPs. Naturally, the presence of magnesium chelators in the reaction can affect the optimal magnesium concentration. A common source of magnesium ion is MgCl₂.

### III. Methods of Producing Reagent Beads

The beads are produced by forming a bead buffer formulation (containing the excipient and biological reagent), creating the beads from the bead buffer formulation, and finally freeze-drying the beads. The produced bead can possess a variety of morphologies and shapes. Exemplary shapes include spherical, near spherical, elliptical or round structures. Exemplary morphologies include smooth or slightly roughened surfaces.

A. Preparation of Reagent Beads

1. Bead Formation

The reagent spheres of the present invention are prepared from reagents suitable for any of the protein based analytical assays of the invention. Typically, an aqueous solution comprising the reagents is prepared. To ensure uniform composition of the reagent spheres, the solution is made homogeneous and all constituents are fully dissolved or in suspension. The final volume per drop of the reagent emulsion is often small, between 2-20 μL, to allow a working volume of 5-200 μL when the lyophilized bead is dissolved in a working solution.

The drops are uniform and precisely measured so that the resulting dried reagent spheres have uniform mass. Using a volumetric or gravimetric dispensing system such as those made by FMI or IVEC has been shown to work well. A time/pressure method such as that used to dispense adhesives also works well.

When the drops are uniform and precisely measured, the imprecision of the mass (coefficient of weight variation) of the reagent spheres prepared from the drops is less than about 3%, and preferably between about 0.3% and about 2.5%. To further decrease the weight variation, the aqueous solution may be degassed using a vacuum pump or vacuum line before the drops of solution are dispensed.

Individual drops of the solution are formed into beads either by dropping the dispensed emulsion onto a cryogenic liquid or onto a cryogenically cooled solid surface, or alternatively, by first dispensing the emulsion a drying surface that facilitates bead formation before the bead is frozen. The composition and shape of such a drying surface determines the drop shape as well as the ease of release from the surface after drying. In preferred embodiments, the dispensed emulsion is placed upon an anodized aluminum pan. Other possible surfaces include glass, polystyrene, wax paper, or Delrin.

Bead formation can also occur by dropping the dispensed emulsion onto a cryogenic liquid or onto a cryogenically cooled solid surface. Cryogenic is defined as a liquefied or solidified gas having a normal boiling or sublimation point below about −75° C.; in some cases, this point is below about −150° C. In an exemplary embodiment, the cryogenic material is nitrogen, Freon, or carbon dioxide. The frozen beads are recovered and then freeze dried to a moisture content of less than about 10%. In some cases, the moisture content is less than 3%.

2. Bead Lyophilization

Lyophillization is extremely useful for enhancing the shelf life and stability o biologicals that are thermolabile and/or unstable in aqueous solution. Vacuum drying, desiccant drying, and freeze-drying of the biological reagent droplets can be utilized for drying the bead material. A standard freeze-drier (such as a VirTis GENESIS) with a control modified to allow operation at partial vacuums can be used.

As noted above, the product to be made using lyophilization is prepared as an aqueous solution or suspension, formed into drops then cooled rapidly to a predetermined temperature that often approaches −50° C. The frozen masses are then lyophilized by methods known in the art, to produce the reagent spheres. The freezing chamber is sealed and the frozen material subjected to heat under high vacuum conditions. The liquid portion sublimes, leaving the desired solid material.

Typically, the frozen drops are lyophilized for about 4 hours to about 24 hours at about 50 to about 450 mTorr, preferably, about 20 hours at about 100 mTorr. The final reagent spheres typically comprise less than about 6% residual moisture, preferably less than about 3%. Reabsorbtion of moisture can occur after lyophilization, necessitating quick removal from the chamber to conditions of low humidity environment. The dried material is porous upon sublimation of ice crystals. This surface character influences the rate of moisture reabsorbtion, dissolution in solution, and shelf life of the dried product.

2. Stability Testing Protein Reagents in the Dry Storage State

While most specific-binding proteins function in the aqueous state in nature, the dry or frozen state is much preferred for stable storage. However, removal of solvent from protein molecules through drying—or through other phase changes such as precipitation and freezing—puts stress on the functional conformation of proteins. Thus, stability of the protein reagent under storage conditions must be evaluated in order to estimate the shelf life of the reagent.

In general, stability testing measures the ability of a product to retain its biological activity up to and beyond its predicted expiration date. Factors affecting the inherent stability of a protein reagent include natural degradation of the protein, resistance to microbial or fungal intrusion, reactivity with excipients, impurity levels imparted by the manufacturing process, and response to the stresses of heat, humidity, and light. Testing protocols simulate storage conditions, either in real time, or on an accelerated basis. Stability tests determine if a significant change in the biological activity of the formulated reagent bead occurs during storage.

To examine the stability of protein reagents, protein reagents are first formulated into reagent beads according to the methods of the invention. An initial measurement of the biological activity of the reagent beads is made, and then the reagent beads are put into storage under defined sets of storage conditions. For real-time stability testing, samples of the reagent beads are removed for assay at intervals spanning the expected storage period. For accelerated testing certain aspects of the storage conditions are exaggerated and samples are taken at shortened intervals over a storage period that is shortened relative to the real-time storage period.

For example, conditions appropriate for real time testing of reagent beads intended for storage at room temperature, might comprise storage at 25° C. at 5% relative humidity. Beads would be withdrawn from the aliquots of stored reagent beads at 6 month intervals over the expected shelf life of the protein reagent and subjected to biological activity testing.

Accelerated testing conditions for a reagent bead stored at room temperature might be 40° C. at 5% relative humidity. Intervals appropriate to accelerated stability testing could be one month or less intervals over a period of time that is significantly shorter than the expected shelf life of the protein reagent. If a significant change occurs at any time during accelerated testing, then testing at an intermediate storage condition may be conducted. For example, intermediate testing conditions for the reagent bead stored at room temperature might be conducted at 30° C. at 5% relative humidity.

Similarly, conditions appropriate for real-time testing a reagent bead intended for storage in a refrigerator, might comprise storage at 5° C. at 5% relative humidity. Samples could be withdrawn at intervals as described above. Accelerated testing conditions for a reagent bead stored in the refrigerator might be conducted at 25° C. at 5% relative humidity. For a product intended for storage in a freezer not colder than −20° C., the test condition might be −20° C.

Biological activity assays to determine stability of the protein reagent may comprise any suitable assay for evaluating the activity of a protein. Assays may include, but are not limited to assays of biological activity assays such as ELISA, nucleic acid amplification reactions coupled with quantitation of the amplification products, enzyme kinetic measurements and the like. In addition to biological activity assays, physical stability of the protein reagent may also be tested. Methods such as size exclusion chromatography may prove useful in assays of physical stability.

### IV. Determining Activity Level of the Protein Based Assay

A. Measuring Activity Level

The invention provides reagent beads for protein based assays that stabilize proteins in solution, and potentiate their activity. Stabilization of the protein in solution permits a biological reaction to take place in a controlled manner only when substrate is added, and/or when the appropriate conditions of temperature are applied. Thus, the reagent beads of the invention provide a ready means for precisely controlling reaction conditions, thereby reducing costs of routine diagnostic assays by preventing anomalous results that require samples to be re-tested.

Measuring Biological Activity

The activity level of a reaction can be measured by any means known in the art for measuring biological activity. The actual means used for detecting and measuring biological activity will depend on the particular protein based assay being conducted. For example, the biological activity of certain enzyme based assays can be measured by determining the catalytic activity of the enzyme reaction. Catalytic activity can be measured by detecting an increase in the k_{cat} or a decrease in the K_{M} for a given substrate, which may be reflected in an increase in the k_{cat}/K_{M} ratio.

For protein based assays involving amplification of an RNA or DNA template, biological activity can be measured by detecting and quantitating the appearance of amplified product over time.

a. Measuring the Activity Level of the Protein Based Assay when the First Bead is Dissolved in Liquid.

When the first reagent bead comprising the protein stabilization matrix is dissolved in a liquid such as water, the activity level of the resulting solution can be measured. When the bead is combined with all the active reactants so that the assay reaction can proceed, we have the first activity level. The bead may or may not contain all the active ingredients. For a nucleic acid amplification assay, substrate comprising amplification primers and a template may either be present in the bead and released upon dissolution, or may be added separately to the solution formed by dissolution of the first reagent bead. Once a substrate is present, temperature is applied so as to promote nucleic acid amplification. Increases in the production of amplification product can be measured during amplification cycles using real-time PCR, or alternatively, after a sufficient number of amplification cycles are completed, the reaction can be terminated and the amount of product can be detected and quantitated by gel electrophoresis.

b. Measuring Potentiation of the Protein Based Assay when the Potentiation Bead Matrix Bead is Dissolved in the First Solution.

Dissolving the potentiation bead matrix bead in the solution created by dissolution of the protein stabilization matrix in a liquid produces a second solution that potentiates the protein based assay. Potentiation means that the performance of a protein based assay is improved over the basic performance level achievable when only the essential (active) reactants are present in the reaction. Inert ingredients are not considered. Potentiation may be brought about through the addition of reagents such as buffers and salts that are able to enhance the performance of the assay, but which are not required for the assay to take place. Potentiation may result in reactions that are measurably more specific, efficient, robust, or which show greater fidelity than the basic reaction.

For a protein based PCR assay a set of standardized conditions might consist of a 50 μL reaction containing:
5 units Taq polymerase
50 mM Tris.HCL pH 8.3,
2.5 mM magnesium chloride,
100 μM of each of the four dNTPs (G, A, C, T), and
BSA (bovine serum albumin)

Such a mixture provides conditions that permit a PCR reaction to take place upon addition of substrate comprising, for example, 0.25 μM amplification primers and 0.1 μM template, and the application of a temperature cycling protocol.

If 25 mM KCl were added to the reaction mixture, the amplification reaction would proceed more efficiently and with greater fidelity since KCl would facilitate the hybridization of the primers to the template nucleic acid. The more efficient reaction would be expected to produce more product per cycle, and perhaps also better quality product. Thus, the reaction containing 25 mM KCl would be said to potentiate the PCR reaction over the reaction lacking KCl.

Similarly, buffering conditions may be adjusted so as to potentiate a protein based assay.

Thus, the term “potentiate” and its derivatives, may be used to convey a relative meaning. If a particular set of reaction conditions is set as a standard for comparison, then the potentiation of other reactions can be described in terms relative to the standard. For example, the above reaction without KCl would be expected to produce X amount of amplification product upon the addition of substrate and the application of a defined temperature cycling protocol. A reaction containing 25 mM KCl that is treated otherwise identically to the reaction lacking KCl might produce Z amount of product upon completion of the temperature cycling protocol. Assuming more product will be produced when 25 mM KCl is present than when no KCl is present, the reaction containing 25 mM KCl may be said to potentiate the protein based PCR assay Z/X-fold relative to the reaction without KCl. In the case where a standard reaction produces little or no measurable product, the activity level of that reaction can be set to 1, such that the activity levels of other reactions can be expressed in terms that are relative to the standard.

Thus, measuring potentiation of a protein based assay comprises measuring the ability and extent of a protein based assay reaction to go to completion. Potentiation can be measured by determining the activity level of a complete reaction in terms of product produced per unit time.

In an exemplary embodiment dissolving the potentiation bead matrix bead in the solution created by dissolution of the protein stabilization matrix in a liquid produces a second solution that potentiates the protein based assay at least 2-fold. In other exemplary embodiments the protein based assay may be potentiated 2.5-fold, 3-fold, 4-fold, 5-fold, 10-fold or 20-fold.

Measuring pH

In some embodiments dissolution of the reagent beads in water results in solutions whose pH needs to be determined. Therefore the invention provides methods for measuring the pH of a solution.

pH is the inverse logarithm of free hydrogen ion concentration. pH can be measured by any means known in the art, but typically is measured with a pH electrode. A pH electrode measures the potential difference between an indicator electrode, which responds to the activity of hydrogen ion in solution, and a reference electrode whose potential remains constant throughout the course of the potentiometric measurement. This potential difference produced is proportional to the hydrogen ion activity of the sample solution, thus enabling the determination of solution pH.

pH electrodes come in a variety of shapes and sizes so that the pH of a solution of any volume can be measured. For example, the pH of solutions at least several milliliters in volume can readily be measured with the available standard pH electrodes. Where smaller volumes must be measured, a micro-pH electrode which measures samples as small as 0.5 μl can be used.

In one embodiment, dissolving one lyophilized reagent bead in liquid yields a first solution of a first pH, and dissolving a second paired lyophilized reagent bead in liquid yields a second solution of a second pH wherein the difference in pH between the first solution, and the second solution is at least 0.2 pH units. In another embodiment, the difference in pH between the first solution, and the second solution is 0.3 pH units. In other embodiments, the difference in pH between the first solution, and the second solution is at least 0.4 pH units, at least 0.5 pH units, at least 0.6 pH units or more.

V. Using Beads in a Protein Based Assay

A. Measuring Activity Level

1. Enzymatic Assays

Any enzymatic assay known in the art may find benefit by employing the compositions and methods of the invention. Polymerase chain reaction is one such enzymatic assay. Another enzymatic assay that may benefit from the methods of the invention are assays which measure tyrosine kinase activity. Some of these assays measure the ability of a tyrosine kinase enzyme to phosphorylate a synthetic substrate polypeptide. For example, an assay has been developed which measures growth factor-stimulated tyrosine kinase activity by measuring the ability of the kinase to catalyze the transfer of the gamma-phosphate of ATP to a suitable acceptor substrate.

2. Antibody Based Assays

Antibody assays for analyses of body fluids, such as blood, plasma, and urine, to diagnose diseases may also benefit from the compositions and methods of the invention. The ELISA assay has been known in the art as one method for analyzing constituents generally present in a small amount in the body fluids. Thus, the methods of the invention provide reagent bead suitable for use in such assays.

3. Receptor Based Assay

The reagent beads and methods of the invention can be used to the benefit of receptor based assays. Receptor binding assays, wherein a receptor protein is mixed with and allowed to bind to a labeled ligand, can benefit from the methods and compositions of the invention. For example, chemokines are a large family of chemotactic cytokines involved in inflammatory, autoimmune and infectious diseases. Through their interaction with G-protein-coupled receptors, chemokines influence many aspects of the immune response. They facilitate leukocyte migration and positioning; dendritic cell function; T cell differentiation; and virus entry, including HIV-1. Therefore, screening libraries of chemical compounds to find drug candidates that modulate specific receptor-ligand interactions, and consequently the cellular events associated with certain pathological conditions are important assays in the development of therapeutic agents. The methods and compositions of the invention may be used to facilitate the discovery of agents that bind chemokine receptors.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill will readily recognize a variety of noncritical parameters which could be changed or modified to yield essentially similar results.

### Example 1

### Making Reagent Beads

I. Lyophilization Formulations

To test the stabilization and potentiation properties of various lyophilization formulations, two sets of lyophilization buffers were prepared. The first set of lyophilization buffers employs separate buffers for the enzyme and for the assay specific reagents (potentiation bead). The buffers are distinguished by the pH and the molarity of the buffering agent.

The second lyophilization buffer set is a single universal buffer formulated for use with both the enzyme and with the assay specific reagents.

Table 1 provides the formulation for the lyophilization buffer used to prepare the protein stabilization matrix for the enzyme reagent. Table 2 provides the formulation for the lyophilization buffer used to prepare the potentiation bead matrix comprising the assay specific reagents.

TABLE 1
Lyophilization Buffer for Enzyme Reagent pH 7.15 Formulation
To this formulation the appropriate components are added

| | | | | 4X Lyophilization |
|---|---|---|---|---|
| | | 4X Lyophilization | | Concentration |
| Component | Vendor/Part # | Concentration | | (gm/100 mL) |
| HEPES Salt (MW = 260.3) | Sigma H3784 | 17.5 | mM | 0.456 |
| HEPES Acid (MW = 238.3) | Sigma H4034 | 14.5 | mM | 0.346 |
| KCl (FW = 74.55) | Sigma P9541 | 60.0 | mM | 0.447 |
| MgCl2 (FW = 95.21)* | Sigma M8266 | 24.0 | mM | 0.229 |
| BSA | Sigma A7638 | 0.18-0.36% | w/v | 0.18-0.36 |
| MIT | Sigma M6045 | 0.1% | w/v | 0.10 |
| Mannitol | Sigma M9546 | 11.0% | w/v | 11.0 |
| Dextran T-40 | AmershemPharmacia | 2.5% | w/v | 2.50 |
| Tween 20. | Pierce #28320 | 0.2% | v/v | 2.0 mL of 10% |
| | | | | stock |
| Antifoam SE-15. | Sigma A8582 | 0.024% | v/v | 0.24 mL of 10% |
| | | | | stock |
| (pH 7.15 ± 0.1) | | | | |

*MgCl₂ concentration can be optimized for a specific assay

TABLE 2
Lyophilization Buffer for target specific reagent pH 8.35 Formulation
To this formulation the appropriate components are added

| | | | | 4X Lyophilization |
|---|---|---|---|---|
| | | 4X Lyophilization | | Concentration |
| Component | Vendor/Part # | Concentration | | (gm/100 mL) |
| HEPES Salt (MW = 260.3) | Sigma H3784 | 117.0 | mM | 3.046 |
| HEPES Acid (MW = 238.3) | Sigma H4034 | 8.0 | mM | 0.191 |
| KCl (FW = 74.55) | Sigma P9541 | 60.0 | mM | 0.447 |
| MgCl2 (FW = 95.21)* | Sigma M8266 | 24.0 | mM | 0.229 |
| BSA | Sigma A7638 | 0.18-0.36% | w/v | 0.18-0.36 |
| MIT | Sigma M6045 | 0.1% | w/v | 0.10 |
| Mannitol | Sigma M9546 | 11.0% | w/v | 11.0 |
| Dextran T-40 | Amersham Pharmacia | 2.5% | w/v | 2.50 |
| Tween 20. | Pierce #28320 | 0.2% | v/v | 2.0 mL of 10% |
| | | | | stock |
| Antifoam SE-15. | Sigma A8582 | 0.024% | v/v | 0.24 mL of 10% |
| | | | | stock |
| (pH 8.35 ± 0.1) | | | | |

*MgCl₂ concentration can be optimized for the specific assay

The lyophilization buffer for the enzyme reagent will contain Taq polymerase enzyme and dNTP's. The lyophilization buffer for the assay specific reagent will contain the primers, fluorescent probes, internal control DNA, and other necessary components.

The reaction pH is controlled by the buffering capacity of the assay specific reagent (ASR) buffer. The HEPES buffer concentration is much higher in the assay specific reagent (125.0 mM) than in the enzyme buffer (32.0 mM). When the HEPES buffer from assay specific reagent and the enzyme reagents are mixed together a final PCR reaction pH of 8.00 is obtained, which is favorable for the PCR reaction.

In order to compare the properties of reagents stabilized in different lyophilization formulas, a universal lyophilization buffer formulation was prepared so that both the enzyme and the assay specific reagent (ASR) could be formulated into beads starting with a pH 8.00 buffer.

Formulation of the universal lyophilization buffer formulation is shown in Table 3. The formulation comprises 100 mM HEPES, pH 8.00±0.1. The appropriate active components are added to this buffer formulation in preparing the enzyme and assay specific reagents.

TABLE 3
Lyophilization Buffer for Enzyme and ASR pH 8.00 Formulation
To this formulation the appropriate components are added

| | | | | 4X Lyophilization |
|---|---|---|---|---|
| | | 4X Lyophilization | | Concentration |
| Component | Vendor/Part # | Concentration | | (gm/100 mL) |
| HEPES Salt (MW = 260.3) | Sigma H3784 | 83.0 | mM | 2.16 |
| HEPES Acid (MW = 238.3) | Sigma H4034 | 17.0 | mM | 0.405 |
| KCl (FW = 74.55) | Sigma P9541 | 60.0 | mM | 0.447 |
| MgCl2 (FW = 95.21)* | Sigma M8266 | 24.0 | mM | 0.229 |
| BSA | Sigma A7638 | 0.18-0.36% | w/v | 0.18-36 |
| MIT | Sigma M6045 | 0.1% | w/v | 0.10 |
| Mannitol | Sigma M9546 | 11.0% | w/v | 11.0 |
| Dextran T-40 | Amershem Pharmacia | 2.5% | w/v | 2.50 |
| Tween 20. | Pierce #28320 | 0.2% | v/v | 2.0 mL of 10% |
| | | | | stock |
| Antifoam SE-15. | Sigma A8582 | 0.024% | v/v | 0.24 mL of 10% |
| | | | | stock |
| (pH 8.00 ± 0.1) | | | | |

*MgCl₂ concentration can be optimized for the specific assay

All the lyophilization buffers mentioned above are a 4× concentrate. A 100 μL final reaction volume requires 12.5 uL of enzyme reagent (which contains lyophilization buffer, enzyme, and dNTP's) and 12.5 uL of assay specific reagent (which contains lyophilization buffer, primers, probes, and internal control DNA, etc.) and 75 μL water containing plus the sample.

In preparing the lyophilized beads the lyophilization buffer is prepared at only 72% of its final volume in order to compensate for volume displacement which will occur as a result of other liquid components are added later. Addition of other components such as dNTP's to the enzyme reagent, and primers and probes for the assay specific reagent dictate the final volume required to give the desired bead size.

### Example 2

### Evaluating the Stability of Protein Reagents for PCR Assay

II. Stability of Ba 4-Plex Reagents

Further experiments tested the stability of the reagent formulations in multiplex PCR reactions involving three or more target templates. A “fourplex” assay was carried out to make this determination. The fourplex assay was developed at Cepheid (Hoffmaster et al. (2002) Emerging Infective Diseases vol. 8:1178-1181).

The fourplex assay involves specific detection of two virulence plasmids from Bacillus anthracis, pXO1 and pXO2, and simultaneous specific detection of two internal controls. Target probes to pXO1 and pXO2, were labeled with FAM (6-carboxy-fluorescein phosphoramidite, pXO1) and LIZ (pXO2) dyes and the internal control probes were labeled with ROX and VIC.

The 4-Plex Reagent stability was established by comparison of the two sets of formulations described above. Stability of reagents was tested based on storage of the reagents at accelerated temperatures. The real-time reagent beads stability (25° C. and 4° C. storage) is ongoing and the data are not shown.

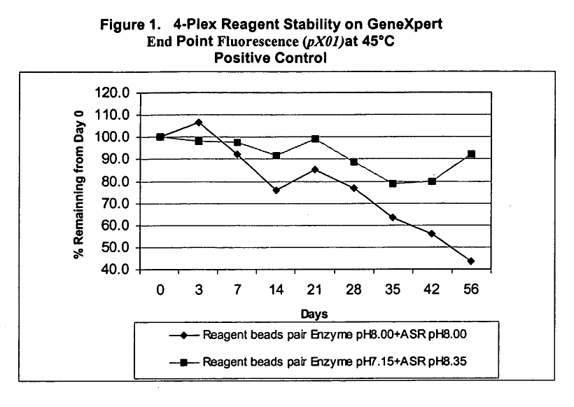

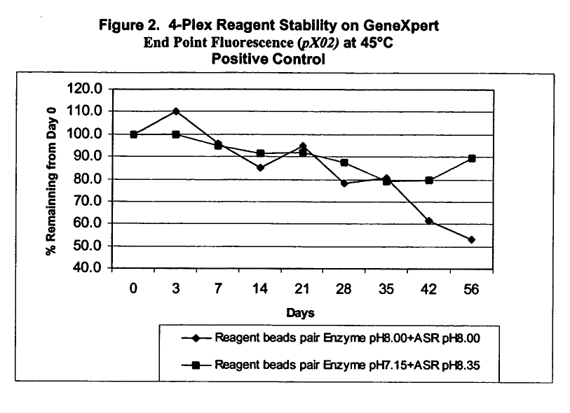

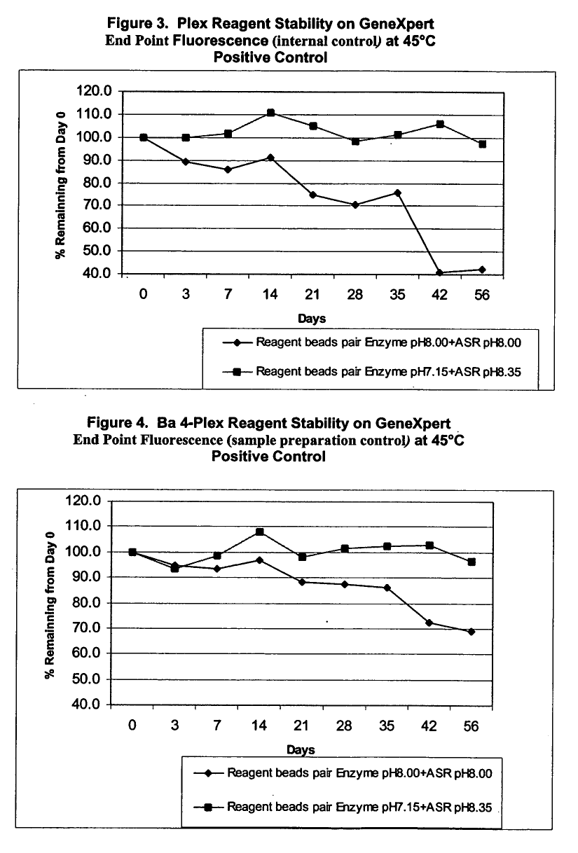

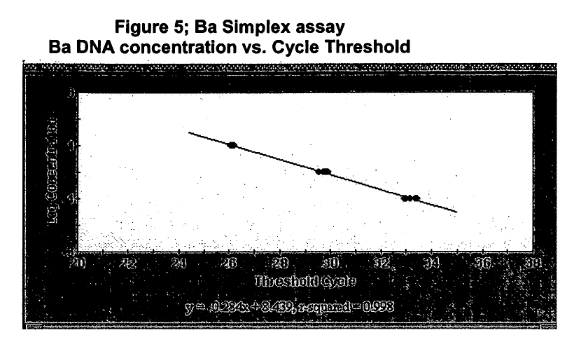

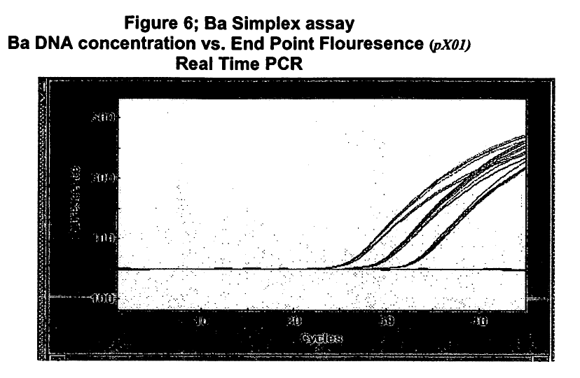

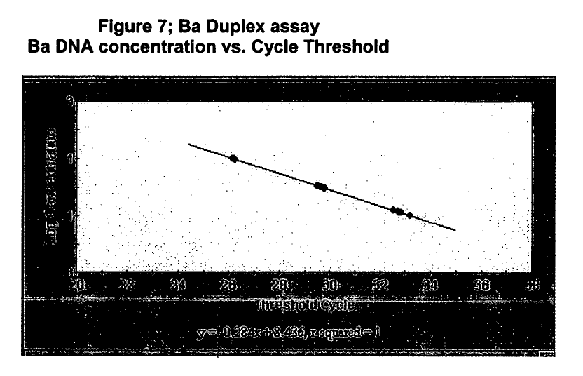

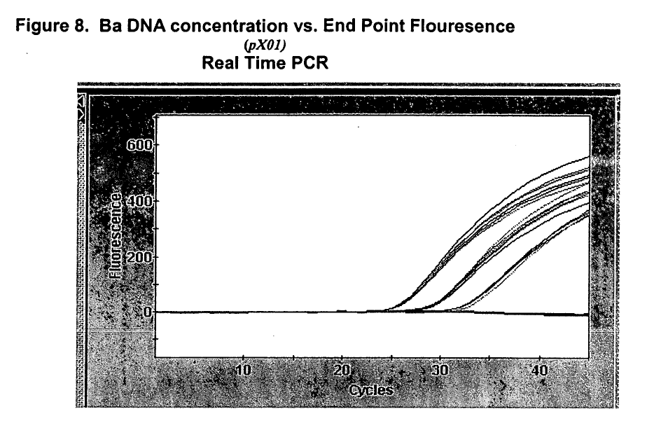

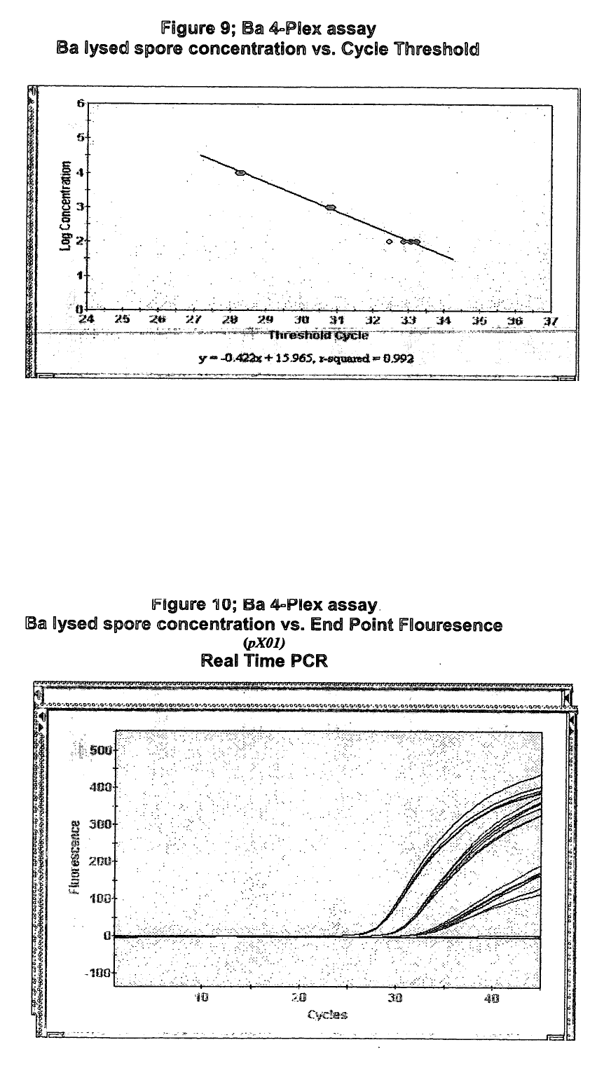

Reagent beads were filled into GeneXpert cartridges and placed in storage temperatures. At least 2 replicates each of negative and positive controls were assayed at each time point. The average of end point fluorescent was calculated and the results are presented. Table 4 shows the results of 35° C. storage for 81 days and Table 5 shows the results of 45° C. storage for 56 days. As can be seen in Tables 4 and 5, the reagent pair with assay specific reagent formulated at pH 8.35±0.1 and enzyme reagent formulated at pH 7.15±0.1 had greater activity remaining after storage, than the reagents that were prepared with pH 8.00±0.1. FIGS. 1 through 4 present the stability results obtained with 45° C. storage, corresponding to Table 5, in graphic form.

TABLE 4
4-Plex Assay: Reagent Stability 35° C. 81 Day results
Positive Control % Activity Remaining From Day 0

| | Both assay | |
|---|---|---|
| | specific reagents | assay specific reagent pH |
| Multiplex Probes | and Enzyme | 8.35 ± 0.1 |
| Fluorescent | Reagents | Enzyme Reagent |
| End Point | pH 8.00 ± 0.1 | pH 7.15 ± 0.1 |
| End Point Fluorescence | 61.5% | 83.2% |
| Target #1 (pX01) | | |
| End Point Fluorescence | 72.5% | 88.2% |
| Target #2 (pX02) | | |
| End Point Fluorescence | 68.8% | 94.7% |
| Internal Control | | |
| End Point Fluorescence | 86.0% | 92.5% |
| Sample preparation Control | | |

TABLE 5
4-Plex Assay: Reagent Stability 45° C. 56 Day Results
Positive Control % Activity Remaining From Day 0

| Multiplex Probes | ASR and Enzyme | ASR pH 8.35 ± 0.1 |
|---|---|---|
| Fluorescent | Reagents | Enzyme Reagent |
| End Point | pH 8.00 ± 0.1 | pH 7.15 ± 0.1 |
| End Point Fluorescence | 43.6% | 92.0% |
| (pX01) | | |
| End Point Fluorescence | 53.2% | 89.5% |
| (pX02) | | |
| End Point Fluorescence | 42.4% | 97.7% |
| Internal Control | | |
| End Point Fluorescence | 69.1% | 96.4% |
| Sample preparation Control | | |

### Example 3

### Carrying Out PCR Assay with Reagent Beads

III. PCR Examples Materials and Instruments

Assay Protocols:

All the assays were run on Cepheid Smart Cyclere, Cepheid Inc., Sunnyvale, Calif. using software v2.0c: S/N 200019, 200016, 900039, 900339, and 900211

Computers S/N: 8BDW021, 23WSG31

Ba Lysed spores or DNA

Enzyme; Ampli Taq lot #E01902 (Roche)+hot start antibody TAKARA (lot #N1803-1)

Cepheid Assay specific primers and fluorescent probes

Procedures

Six replicates for each sample containing 0 (negative control), 0.1 pg, 1.0 pg, 10.0 pg, Ba DNA/25 μL reaction was assayed for the simplex and duplex assays.

Simplex assays comprise only one template-primer-probe set, and duplex assays comprise two primer and probe sets.

And six replicates of samples containing 0 (Negative control), 4×10², 4×10³, 4×10⁴ lysed Ba spores per 85 uL reaction were assayed for the 4-Plex assay.

ASSAY PROTOCOL ON SMART CYCLER ® SOFTWARE V2.0C

| | | |
|---|---|---|
| Step 1 | 95° C., 30 seconds | Optics off |
| Step 2 | 95° C., 1 second | Optics off |
| 45 cycles | 65° C., 20 seconds | Optics on |

For each reaction the cycle threshold (Ct), and the end point fluorescence (EP) were measured. The cycle threshold (Ct), correlates with the log-linear phase of PCR amplification and is the first cycle in which there is significant increase in fluorescence above the background.

TABLE 6
Ba Simplex and Duplex Assays
Cycle Threshold and End point fluorescence with target DNA

| DNA concentrate | Ba Simplex Assay | | Ba Duplex Assay | |
|---|---|---|---|---|
| on (pg/25 uL | Cycle | End Point | Cycle | End Point |
| reaction) | Threshold | Flouresence | Threshold | Flouresence |
| 0.0 | 0.0 | −0.2 | 0.0 | −6.8 |
| 0.1 | 32.7 | 343.2 | 32.3 | 362.0 |
| 1.0 | 29.2 | 405.2 | 28.9 | 433.4 |
| 10.0 | 25.8 | 421.6 | 25.4 | 509.2 |

Each value represents an average of six replicates

TABLE 7
Ba 4-Plex Assays
Cycle Threshold and End point fluorescence with target DNA

| | Target #1 (pX01) | | Target #2 (pX02) | |
|---|---|---|---|---|
| Ba Lysed spores/ | Cycle | End Point | Cycle | End Point |
| 85 uL reaction | Treshold | Flouresence | Treshold | Flouresence |
| 0.0 | 0.0 | 0.2 | 0.0 | 9.7 |
| 400 | 34.22 | 158.2 | 33.66 | 184.5 |
| 4,000 | 30.48 | 351.2 | 30.29 | 299.6 |
| 40,000 | 27.74 | 402.2 | 27.43 | 354.9 |

Each value represents an average of six replicates

What is claimed is:

1. A multi-bead assay system for a protein based assay comprising:
a first bead comprising protein and a protein stabilization matrix that forms a first solution when dissolved in liquid, wherein the first solution permits a first activity level for the assay said first activity level greater than zero; and
a second bead comprising a potentiation bead matrix that when dissolved in the first solution forms a second solution that potentiates the protein based assay to achieve a second activity level that is higher than the first activity level.

2. The multi bead assay system of claim 1, wherein, the second solution potentiates the protein based assay at least 2-fold over the first activity level of the first solution.

3. The multi bead assay system of claim 2, wherein, the second solution potentiates the protein based assay 5-fold over the first activity level of the first solution.

4. The multi bead assay system of claim 1, wherein the protein based assay is selected from the group consisting of an enzymatic assay, an antibody based assay, and a receptor based assay.

5. The multi bead assay system of claim 1, wherein the protein based assay comprises nucleic acid amplification, the first bead comprises a lyophilized reagent bead containing at least one enzyme for the nucleic acid amplification, and the second bead comprises a lyophilized reagent bead containing primers for amplification of at least one analyte nucleic acid sequence.

6. The multi bead assay system of claim 5, wherein the second bead further comprises at least one probe for detecting the analyte nucleic acid sequence.

7. The multi bead assay system of claim 1, wherein the protein based assay comprises nucleic acid amplification, the first bead comprises a lyophilized reagent bead containing at least one enzyme for the nucleic acid amplification, and the second bead comprises a lyophilized reagent bead containing primers for amplification of at least two analyte nucleic acid sequences.

8. The multi bead assay system of claim 7, wherein the second bead further comprises probes for detecting the analyte nucleic acid sequences.

9. The multi bead assay system of claim 1, wherein the protein based assay comprises nucleic acid amplification, the first bead comprises a lyophilized reagent bead containing at least one enzyme for the nucleic acid amplification, and the second bead comprises a lyophilized reagent bead containing primers for amplification of at least three analyte nucleic acid sequences.

10. The multi bead assay system of claim 9, wherein the second bead further comprises probes for detecting the analyte nucleic acid sequences.

11. A multi-bead assay system for a protein based assay, the multi-bead assay system comprising a first bead and a second bead, wherein the first bead yields a first solution of a first pH when the first bead is dissolved in liquid, the second bead yields a second solution of a second pH when the second bead is dissolved in liquid, and the difference in pH between the first solution and the second solution is at least 0.4 pH units.

12. The multi-bead assay system of claim 11, wherein combining the first bead and the second bead in liquid yields a third solution of a third pH that permits a protein based assay to take place at an activity level that is greater than an activity level of the protein based assay at the first pH.

13. The multi bead assay system of claim 11, wherein the protein based assay comprises nucleic acid amplification, the first bead comprises a lyophilized reagent bead containing at least one enzyme for the nucleic acid amplification, and the second bead comprises a lyophilized reagent bead containing primers for amplification of at least one analyte nucleic acid sequence.

14. The multi bead assay system of claim 13, wherein the second bead further comprises at least one probe for detecting the analyte nucleic acid sequence.

15. The multi bead assay system of claim 11, wherein the protein based assay comprises nucleic acid amplification, the first bead comprises a lyophilized reagent bead containing at least one enzyme for the nucleic acid amplification, and the second bead comprises a lyophilized reagent bead containing primers for amplification of at least two analyte nucleic acid sequences.

16. The multi bead assay system of claim 15, wherein the second bead further comprises probes for detecting the analyte nucleic acid sequences.

17. The multi bead assay system of claim 11, wherein the protein based assay comprises nucleic acid amplification, the first bead comprises a lyophilized reagent bead containing at least one enzyme for the nucleic acid amplification, and the second bead comprises a lyophilized reagent bead containing primers for amplification of at least three analyte nucleic acid sequences.

18. The multi bead assay system of claim 17, wherein the second bead further comprises probes for detecting the analyte nucleic acid sequences.

19. A multi-bead reaction system for nucleic acid amplification comprising:
(i) a first lyophilized reagent bead comprising at least one enzyme for nucleic acid amplification in a protein stabilization matrix; and
(ii) a second lyophilized reagent bead comprising oligonucleotides for nucleic acid amplification in a potentiation bead matrix, wherein dissolving the reagent beads in liquid potentiates the nucleic acid amplification reaction.

20. The multi-bead reaction system for nucleic acid amplification of claim 19, further comprising a means for detecting amplification product.

21. The multi-bead reaction system for nucleic acid amplification of claim 20, wherein the means for detecting amplification product comprises an intercalating agent in the second bead.

22. The multi-bead reaction system for nucleic acid amplification of claim 20, wherein the means for detecting amplification products comprises at least one hybridization probe in the second bead.

23. The multi bead reaction system of claim 19, wherein the second bead comprises primers for amplification of at least one analyte nucleic acid sequence.

24. The multi bead reaction system of claim 23, wherein the second bead further comprises at least one probe for detecting the analyte nucleic acid sequence.

25. The multi bead reaction system of claim 19, wherein the the second bead comprises primers for amplification of at least two analyte nucleic acid sequences.

26. The multi bead reaction system of claim 25, wherein the second bead further comprises probes for detecting the analyte nucleic acid sequences.

27. The multi bead reaction system of claim 19, wherein the the second bead comprises primers for amplification of at least three analyte nucleic acid sequences.

28. The multi bead reaction system of claim 27, wherein the second bead further comprises probes for detecting the analyte nucleic acid sequences.

29. The multi-bead reaction system of claim 19, further comprising a third bead that comprises an oligonucleotide probe.

30. The multi-bead reaction system of claim 19, wherein the nucleic acid amplification reaction is an isothermic amplification reaction.

31. The multi-bead reaction system of claim 30, wherein the isothermic amplification reaction is selected from the group consisting of strand displacement amplification, transcription mediated amplification, rolling circle amplification and nucleic acid sequence based amplification.

32. The multi-bead reaction system of claim 19, wherein the nucleic acid amplification reaction is a thermocyclic amplification reaction.

33. The multi-bead reaction system of claim 32, wherein the thermocyclic amplification reaction is selected from the group consisting of polymerase chain reaction (PCR), reverse transcriptase polymerase chain reaction (RT-PCR), and ligase chain reaction (LCR).

34. A method for performing a protein based assay, the method comprising the steps of:
(a) combining in an aqueous solution:
i) a first bead comprising protein and a protein stabilization matrix that forms a first solution when dissolved in liquid, wherein the first solution permits a first activity level for the assay; and
ii) a second bead comprising a potentiation bead matrix that when dissolved in the first solution forms a second solution that potentiates the protein based assay to achieve a second activity level that is higher than the first activity level; and
(b) allowing the assay to perform.

35. The method of claim 34, wherein the protein is a nucleic acid polymerase selected from the group consisting of a DNA polymerase, an RNA polymerase, and a reverse transcriptase.

36. The method of claim 34, wherein the protein is an enzyme.

37. The method of claim 34, wherein the protein is an antibody.

38. The method of claim 34, wherein the second solution potentiates the protein based assay at least 2-fold over the first activity level of the first solution.

39. The method of claim 34, wherein the protein based assay comprises nucleic acid amplification, the first bead comprises a lyophilized reagent bead containing at least one enzyme for the nucleic acid amplification, the second bead comprises a lyophilized reagent bead containing primers for amplification of at least one analyte nucleic acid sequence, and the step of allowing the assay to perform comprises amplifying the analyte nucleic acid sequence, if present in the solution.

40. The method of claim 39, wherein the second bead further comprises at least one probe for detecting the analyte nucleic acid sequence, and the method further comprises the step of detecting the analyte nucleic acid sequence, if present.

41. The method of claim 34, wherein the protein based assay comprises nucleic acid amplification, the first bead comprises a lyophilized reagent bead containing at least one enzyme for the nucleic acid amplification, the second bead comprises a lyophilized reagent bead containing primers for amplification of at least two analyte nucleic acid sequences, and the step of allowing the assay to perform comprises amplifying the analyte nucleic acid sequences, if present in the solution.

42. The method of claim 41, wherein the second bead further comprises probes for detecting the analyte nucleic acid sequences, and the method further comprises the step of detecting the analyte nucleic acid sequences, if present.

43. A method for performing a protein based assay, the method comprising the steps of:
a) combining first and second beads in an aqueous solution, wherein the first bead comprises a bead that yields a first solution of a first pH when the bead is dissolved in liquid, the second bead comprises a bead that yields a second solution of a second pH when the bead is dissolved in liquid, and the difference in pH between the first solution and the second solution is at least 0.4 pH units; and
b) allowing the assay to perform.

44. The method of claim 43, wherein the protein based assay comprises nucleic acid amplification, the first bead comprises a lyophilized reagent bead containing at least one enzyme for the nucleic acid amplification, the second bead comprises a lyophilized reagent bead containing primers for amplification of at least one analyte nucleic acid sequence, and the step of allowing the assay to perform comprises amplifying the analyte nucleic acid sequence, if present in the solution.

45. The method of claim 44, wherein the second bead further comprises at least one probe for detecting the analyte nucleic acid sequence, and the method further comprises the step of detecting the analyte nucleic acid sequence, if present.

46. The method of claim 43, wherein the protein based assay comprises nucleic acid amplification, the first bead comprises a lyophilized reagent bead containing at least one enzyme for the nucleic acid amplification, the second bead comprises a lyophilized reagent bead containing primers for amplification of at least two analyte nucleic acid sequences, and the step of allowing the assay to perform comprises amplifying the analyte nucleic acid sequences, if present in the solution.

47. The method of claim 46, wherein the second bead further comprises probes for detecting the analyte nucleic acid sequences, and the method further comprises the step of detecting the analyte nucleic acid sequences, if present.

48. The method of claim 43, wherein the protein based assay is an enzymatic assay.

49. The method of claim 43, wherein the assay is an immunoassay.

50. The method of claim 43, where the assay is a polymerase chain reaction (PCR) assay.

51. The method of claim 43, wherein the assay is a reverse transcriptase polymerase chain reaction (RT-PCR) assay.
